(19) United States
(12) Patent Application Publication
(10) Pub. No.: US 2010/0305466 A1
(43) Pub. Date: Dec. 2, 2010

(54) INCENTIVE SPIROMETRY AND NON-CONTACT PAIN REDUCTION SYSTEM

(75) Inventor: Stephen B. CORN, Sharon, MA (US)

Correspondence Address: LAHIVE & COCKFIELD, LLP;FLOOR 30, SUITE 3000, ONE POST OFFICE SQUARE, BOSTON, MA, 02109, US

(73) Assignee: ENGINEERED VIGILANCE, LLC, Sharon, MA (US)

(21) Appl. No.: 12/829,346

(22) Filed: Jul. 1, 2010

Related U.S. Application Data
(63) Continuation-in-part of application No. 11/308,675, filed on Apr. 20, 2006.
Application No. 11/308,675 is a continuation-in-part of application No. 12/363,467, filed on Jan. 30, 2009.
(60) Provisional application No. 61/269,897, filed on Jul. 1, 2009.
(60) Provisional application No. 61/062,849, filed on Jan. 30, 2008.
(60) Provisional application No. 60/672,681, filed on Apr. 20, 2005.
(60) Provisional application No. 60/672,600, filed on Apr. 20, 2005.
(60) Provisional application No. 60/672,678, filed on Apr. 20, 2005.
(60) Provisional application No. 60/672,680, filed on Apr. 20, 2005.
(60) Provisional application No. 60/672,659, filed on Apr. 20, 2005.

(51) Int. Cl. A61B 5/087

(52) U.S. Cl. 600/538

(57) ABSTRACT

A non-contact mechanism for encouraging and facilitating incentive spirometry, ensuring that it is performed adequately, in a timely manner, and for a sufficient duration is discussed. The embodiments also quantitatively and qualitatively keep a record of the incentive spirometry activity, including recording the performance in an electronic medical record. A non-contact monitoring system is used to generate a breathing waveform for a subject that may be compared to target waveforms. Visual and non-visual cues may then be provided to the subject to help guide the subject towards the desired breathing pattern.

### RELATED APPLICATIONS

The present application is related to and claims the benefit of: U.S. Provisional Patent Application No. 61/269,897, filed Jul. 1, 2009, entitled “Method and Apparatus: Incentive Spirometry System (ISS) and Non-Contact Pain Reduction Technology”. This application is also a continuation-in-part of U.S. patent application Ser. No. 11/308,675, filed Apr. 20, 2006, entitled “Method for Using a Non-Invasive Cardiac and Respiratory Monitoring System”, which is related to and claims the benefit of U.S. Provisional Patent Application No. 60/672,678, filed Apr. 20, 2005, entitled “Medicine's First In-Home, Evidence-Based Medication Response System”, U.S. Provisional Patent Application No. 60/672,600, filed Apr. 20, 2005, entitled “Smart Infant Monitor and Effector ‘Watch Band’ Technology”, U.S. Provisional Patent Application No. 60/672,659, filed Apr. 20, 2005 entitled “Hand-Held Non-Contact Heart Rate and Respiratory Rate Monitor”, U.S. Provisional Patent Application No. 60/672,680 filed Apr. 20, 2005, entitled “Non-Contact Heart Rate and Rhythm Detection”, and U.S. Provisional Patent Application No. 60/672,681, filed Apr. 20, 2005 entitled “Neuro-Degenerative Monitoring System.” This application is also a continuation-in-part application of U.S. patent application Ser. No. 12/363,467, filed Jan. 30, 2009, entitled “System and Method Providing Biofeedback for Anxiety and Stress Reduction”, which claims the benefit of U.S. Provisional Patent Application No. 61/062,849, filed Jan. 30, 2008, entitled “Biofeedback and Anxiety/Stress Reduction Method and Device”. Each of the above-referenced applications is incorporated by reference herein in their respective entireties.

### BACKGROUND

Incentive spirometry is an important component of medical care, especially in the post-operative period. The technique of incentive spirometry was first developed to help bronchial hygiene before and after surgery. It was observed that many patients who underwent surgery developed fever and lung collapse (atelectasis) after the first few days of surgery. This was due to a combination of pain, lack of a cough reflex and continued shallow breathing. The degree of lung collapse suffered by post-operative patients is variable as some individuals only develop mild atelectasis accompanied by a fever. In other individuals atelectasis can be quite severe and compromise oxygenation of the lung. For this reason incentive spirometry was developed to encourage patients to take deep and slow breaths to assist in expansion of the lungs after surgery.

Conventionally incentive spirometry has been accomplished by the use of a device that provides the patient with a visual feedback when they inhale for a minimum of 1-3 seconds. The primary goal of the procedure is to increase the lung volumes and improve the performance of the respiratory muscles so that the entire lung expands. When the procedure is performed on a regular basis after surgery, the smaller airways remain open and collapse of the lungs is prevented. The types of surgery that commonly cause lung collapse include incisions on the chest, upper abdomen, or on patients who smoke or have obstructive lung disease. Additionally, incentive spirometry is today used on many non-surgical patients. For example, incentive spirometry may benefit some bed-ridden patients or those who are paralyzed and who have also developed weakened respiratory muscles and are therefore prone to the development of atelectasis. Incentive spirometry is now also widely used by patients in the intensive care units, extended care facilities, long-term home care and on general medical floors.

### BRIEF SUMMARY

Embodiments of the present invention provide a non-contact mechanism for encouraging and facilitating incentive spirometry, ensuring that it is performed adequately, in a timely manner, and for a sufficient duration. The embodiments also quantitatively and qualitatively keep a record of the incentive spirometry activity, including recording the performance in an electronic medical record. A non-contact monitoring system is used to generate a breathing waveform for a subject that may be compared to target waveforms. Visual and non-visual cues may then be provided to the subject to help guide the subject towards the desired breathing pattern.

In one embodiment, a non-contact monitoring system for monitoring incentive spirometry exercises includes a respiratory waveform detection module. The respiratory waveform detection module performs non-contact monitoring of a subject performing incentive spirometry exercises and generates a waveform based on detected respiratory motion of the subject. The system also includes an analysis module that programmatically analyzes the generated waveform based on a stored target waveform indicative of a target respiratory motion for an incentive spirometry exercise. The system further includes a biofeedback module that provides biofeedback to the subject to assist the subject in obtaining or maintaining the target waveform. The biofeedback is based on a result of the analyzing of the generated waveform.

In another embodiment, an integrated non-contact monitoring apparatus for monitoring incentive spirometry exercises includes a respiratory waveform detection module. The respiratory waveform detection module performs non-contact monitoring of a subject performing incentive spirometry exercises and generates a waveform based on detected respiratory motion of the subject. The apparatus also includes an analysis module that programmatically analyzes the generated waveform based on a stored target waveform indicative of a target respiratory motion for an incentive spirometry exercise. The apparatus further includes a biofeedback module that provides biofeedback to the subject to assist the subject in obtaining or maintaining the target waveform. The biofeedback is based on a result of the analyzing of the generated waveform. A display surface that is used to provide the biofeedback in the form of a display of the generated waveform and/or the target waveform is also part of the apparatus.

In an embodiment, a method for performing non-contact monitoring of incentive spirometry exercises performs non-contact monitoring of a subject performing an incentive spirometry exercise to detect respiratory motion of the subject. The method generates programmatically a waveform based on the detected respiratory motion and analyzes programmatically the generated waveform based on a stored target waveform indicative of a target respiratory motion for an incentive spirometry exercise. The method also provides biofeedback to the subject to assist the subject in obtaining or maintaining the target waveform. The biofeedback is based on a result of the analyzing of the generated waveform.

In another embodiment, a physical computer-readable medium holds computer-executable instructions that when executed cause one or more computing devices to perform non-contact monitoring of a subject performing incentive spirometry exercises to detect respiratory motion of the subject. The instructions generate programmatically a waveform based on the detected respiratory motion and analyze programmatically the generated waveform based on a stored target waveform indicative of a target respiratory motion for an incentive spirometry exercise. The instructions when executed further provide biofeedback to the subject to assist the subject in obtaining or maintaining the target waveform. The biofeedback is based on a result of the analyzing of the generated waveform.

### BRIEF DESCRIPTION OF THE DRAWINGS

The accompanying drawings, which are incorporated in and constitute a part of this specification, illustrate one or more embodiments of the invention and, together with the description, explain the invention. In the drawings:

FIG. 1 depicts an exemplary environment suitable for practicing embodiments of the present invention;

FIG. 2 depicts an exemplary integrated non-contact monitoring apparatus;

FIG. 3 depicts an exemplary sequence of steps performed by an embodiment of the present invention to analyze the performance of an incentive spirometry exercise; and

FIG. 4 depicts an exemplary waveform that may be displayed to a user to assist the user in learning breathing techniques for performing incentive spirometry.

### DETAILED DESCRIPTION

Though incentive spirometry is known to be of benefit for post-operative pulmonary care, compliance with post-operative incentive spirometry regimens has been shown to be low. This lack of compliance occurs for a number of reasons. Even though incentive spirometry is beneficial for most medical patients, there are some patients who may find performing the process difficult without supervision while other patients do not receive the necessary training. In some cases, there may be pain associated with incentive spirometry, especially after surgery, and the pain discourages the patient. In rare cases incentive spirometry can exacerbate asthma and lead to fatigue. Additionally, healthcare professionals are busy with very high patient loads. As such, it is difficult for providers to encourage, make interesting, and ensure the procedure is being done correctly. Another factor in low compliance rates is that patients are often receiving pain medications or sedatives and truly forget to perform the exercise. Further, with visitors, trips for procedures and imaging, and other distractions, patients often are interrupted and do not return to perform the exercise. Finally, current incentive spirometry systems are not engaging or interesting and do not provide the feedback for a “job well done”

The embodiments of the present invention provide an incentive spirometry system that encourages and trains patients to perform incentive spirometry with minimal supervision required by health care providers. Patients may be affirmatively prompted to perform incentive spirometry at appropriate times based upon real-time physiological data. The system also provides pain control utilizing a variety of biofeedback techniques that help reduce reliance on post-operative opioids.

One aspect of the incentive spirometry system described herein is the ability of the system to programmatically prompt the patient to perform the activity. Other incentive spirometry devices are passive. The present invention is programmable and allows for pre-selected programs to be run, user or point-of-care programming, and patient prompts based on real-time user physiologic data. That is, the incentive spirometry system can monitor the patient's rate, rhythm, and amplitude of breathing, along with analyzing for cough, sighs and other characteristic breathing waveforms, including breathing frequency analysis (analysis in the frequency domain, fractal analysis, and related chaos theory). As such, the incentive spirometry system can initiate or delay standard therapy based on the physiologic state of the patient. If, for example, the incentive spirometry system detects shallow breathing, the incentive spirometry system can initiate a therapy session tailored to this finding sooner than originally scheduled. On the other hand, should the incentive spirometry system detect deep regular breathing, without sighs, the incentive spirometry system can delay or skip a scheduled session. All of the treatment information can be recorded and stored, transmitted to an electronic medical record (EMR) or transmitted to a healthcare professional for monitoring.

The embodiments of the present invention also lead to better compliance with incentive spirometry regimens as the system can provide encouragement make the experience interesting. The incentive spirometry system can provide visual, audio and tactile feedback to train the individual in the incentive spirometry exercise and encourage the user to continue the exercise. Further, with a superimposed waveform, the background can be either a relaxation session (see below) or entertainment to facilitate the session. Further, if the user is not performing the exercise properly an interruption can be introduced (either visual, audio or tactile or any combination) that instructs the user in the proper method of performing the exercise.

Another feature of the incentive spirometry system is that since the system utilizes non-contact monitoring to measure the breathing of the participant, it does not require the patient to have his or her eyes open to look at water-tubes or other indicators of achievement of goals of the exercise. As such, the user can have his or her eyes closed while performing incentive spirometry. The patient can thus perform relaxation exercises, shown to decrease pain and suffering, while being ensured that he or she is properly and effectively performing the incentive spirometry exercise. The incentive spirometry system can intervene, encourage and instruct the patient while the patient keeps his or her eyes closed. This feature is also of benefit for those patients with limited or no eyesight and for those with cognition co-morbidities.

Post-operative pain control (Relaxation Response) and postoperative wellness (incentive spirometry) is important. Use of post-operative opioids can lead to much morbidity and mortality including respiratory depression (which leads to further atelectasis) and even respiratory arrest and death. Post-operative relaxation exercises may reduce reliance on opioids thereby reducing side-effects from opioids (GI issues, CNS issues such as falls, alertness, etc.) as well as postoperative respiratory arrest. Reducing the effects of opioids on the GI system is a significant factor for the comfort and well being of patients and for reducing costly care associated with these side effects.

The incentive spirometry system provided by the embodiments of the present invention is also truly “incentivized” in that this system records and verifies that the activity was or was not performed by the patient. As such, a patient wishing to be viewed as compliant by his or her healthcare team, family, etc is incentivized to perform the therapy, as there now will be an objective measure indicating whether the exercise was performed or not. This is in marked contrast to blow-bottles or other devices, which are passive and offer no means to verify that the exercise was done, nor a means to store the record of the exercise being done.

As described further below, the incentive spirometry system described herein may be a self-contained unit, may interface with existing hospital and monitoring equipment, or may be self-contained within computing devices such as smartphones, tablet computing devices, PDAs or laptops.

In one embodiment the incentive spirometry system can make use of ultrasound (or audible, laser, radar, or other energies of the electromagnetic spectrum) to emit a signal that is reflected off of the subject and back to the device to provide time-of-flight distance measurements without requiring physical contact with the patient. Different techniques for performing non-contact monitoring are described below. Taking many samples over time yields a breathing waveform that will provide the device, caretakers and patient with breathing rate, rhythm, and amplitude. This information may be analyzed in both the time and frequency domain and provides waveform characteristics such as coughing, sighing and sneezing. Further the device can serve as a breathing monitor and alert the care takers to decreases, increases or other changes in breathing characteristics that may warrant intervention.

In an alternative embodiment, a probe may be attached to the patient, and emit a signal detected by the incentive spirometry system, which in one embodiment may be executing on an iPhone, PDA, Smartphone or similar device.

In various embodiments, the incentive spirometry system may provide a number of features. For example, the incentive spirometry system may provide an idealized waveform with adjustable inspiratory/expiratory (I:E) ratio rate and amplitude (calculation of the I:E ratio is described further below). The incentive spirometry system may also provide a display depicting the patient's waveform in relation to an idealized waveform and may provide an option to turn the depiction of the patient's waveform on and off. A display of the patient's waveform may provide adjustable amplitude “goals”—such as bars to target for inspiration and expiration- and/or an adjustable timer showing how far into the exercise the patient is and how far the patient has to go. The background to the displayed information may be programmatically or manually adjustable to allow a video clip or slideshow designed to create a relaxing atmosphere to play. The incentive spirometry system may also provide a log to show date, time, and time spent doing exercise. Data displayed by the incentive spirometry system may include icons for transmitting the data to an electronic medical record, for transmitting data to a primary care physician or for transmitting data to a rapid response team.

The embodiments of the present invention may advise a patient to follow a displayed waveform to achieve the sufficient depth (amplitude) of breathing over a certain time-period. However, unlike passive systems, this incentive spirometry system indicates to the patient if he or she is achieving the desired depth and exercise, guides the patient to achieve the proper depth, encourages use over a specified timeframe (for example, 5 or 10 minutes or so) and alerts the patient throughout the day that it is time to do the incentive spirometry. The system also documents the performance in an electronic medical record. Visual cues, such as bars or other symbols may be displayed or audio or tactile cues used to show the user that he or she has taken a breath of adequate amplitude or character (inspiratory pauses, I:E ratio) and breaths of appropriate pattern for the exercise.

Rocking motion, regardless of cause, may affect a breathing waveform. In one embodiment, use of an accelerometer, either attached to the individual, or chair (for example a rocking office chair) may be used to filter out the motion artifact and interfaced with the incentive spirometry system. Given that devices such as the iPhone contain accelerometers, motion artifact may be filtered out of the breathing waveform by putting the iPhone or similar device in direct contact with the individual or on the surface, such as the bedding.

In one embodiment of the incentive spirometry system a mouthpiece provides a user with fixed or variable resistance for inhalation and exhalation during the exercise. Further, this mouthpiece may be interfaced with the incentive spirometry system, either through a direct connection, such as to the earpiece port or multi-pin port of an iPhone. Alternatively, it may transmit the information to LAN, Wi-Fi, Bluetooth, networks such as 3G or 4G, or other information transmitting means known to the art and industry.

As noted above, the embodiments of the present invention may use a non-contact monitoring system to monitor a patient's respiration in order to determine whether incentive spirometry exercises are being performed properly and to provide biofeedback to relax the patient or otherwise improve the incentive spirometry exercise. The embodiments of the present invention may utilize a non-contact monitoring system to remotely monitor physiologic functions of a monitored subject. Non-contact measurement of breathing parameters (e.g.: rate, rhythm amplitude, pauses, inspiratory to expiratory ratio, breathing frequency variability) and/or body movements are used in the diagnostic process. Feedback can be provided to the monitored subject in real-time either programmatically or from doctors in remote locations and treatments and therapies may be adjusted as needed.

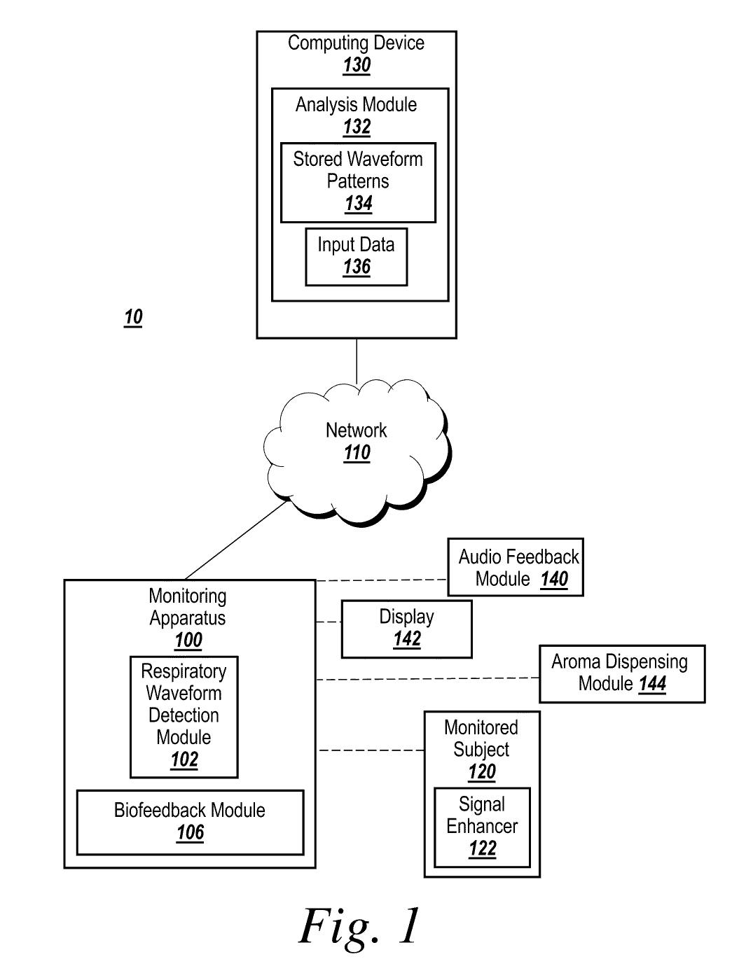

FIG. 1 depicts an exemplary environment suitable for practicing embodiments of the present invention. Monitoring system 10 may include monitoring apparatus 100 that is used to monitor physiological factors for a monitored subject 120. Monitoring apparatus 100 may include a respiratory waveform detection module 102. Respiratory waveform detection module 102 is used to perform non-contact respiratory monitoring of monitored subject 120 and to generate a waveform representing the monitored respiratory process. A number of different techniques to perform the non-contact monitoring may be used and are described in greater detail below.

Once a waveform representing the monitored respiratory function has been generated, monitoring system 10 analyzes the generated waveform to determine whether the patient is properly performing incentive spirometry. In one embodiment, the generated waveform is programmatically analyzed by a software analysis module 132 executing on a computing device 130. Computing device 130 may take many forms, including but not limited to a personal computer, workstation, server, network computer, quantum computer, optical computer, bio computer, Internet appliance, mobile phones and other mobile devices such as smartphones, a pager, a tablet computing device, or other form of digital computer configured to execute analysis module 132. Computing device 130 may be electronic and may include a Central Processing Unit (CPU), memory, storage, input control, modem, network interface, etc. The CPU may control each component of computing device 130 to provide an environment suitable for executing analysis module 132. The memory on computing device 130 temporarily stores instructions and data and provides them to the CPU so that the CPU operates the computing device 130.

Optionally, computing device 130 may include multiple CPUs for executing software loaded in memory and other programs for controlling system hardware. Each of the CPUs can be a single or a multiple core processor. The code loaded in the memory may run in a virtualized environment, such as in a Virtual Machine (VM). Multiple VMs may be resident on a single processor. Also, part of the code could be run in hardware, for example, by configuring a field programmable gate array (FPGA), using an application specific instruction set processor (ASIP) or creating an application specific integrated circuit (ASIC).

Input control for the computing device 130 may interface with a keyboard, mouse, microphone, camera, such as a web camera, or other input devices such as a 3D mouse, space mouse, multipoint touchpad, accelerometer-based device, gyroscope-based device, etc. Computing device 130 may receive, through the input control, input data relevant for calculating target waveforms for monitored subject 120. Optionally, computing device 130 may display data relevant to the generated waveform on a display as part of the analysis process.

In one embodiment, monitoring apparatus 100 communicates with computing device 130 over a network 110. Network 110 may be the Internet, intranet, LAN (Local Area Network), WAN (Wide Area Network), MAN (Metropolitan Area Network), wireless network or some other type of network over which monitoring apparatus 100 and computing device 130 can communicate. Although depicted as a separate device in FIG. 1, it should also be appreciated that computing device 130 may be part of an integrated apparatus with monitoring apparatus 100.

Analysis module 132 analyzes the generated waveform produced by monitoring apparatus 100. The generated waveform is compared against stored waveform patterns 134 to determine whether the current generated waveform is indicative of a patient properly performing incentive spirometry. The selection of the comparison waveform from the stored waveform patterns may utilize previous input data 136 that includes information regarding the monitored subject such as a previously stored base-line breathing waveform, personal medical information (e.g. sex, height, weight, age, family history of various diseases, etc. and occupational information). Based on available data, the analysis module 132 selects either a previously stored base-line breathing waveform, a customized target waveform or a default waveform for comparison to the generated waveform.

In one embodiment, the analysis of the generated waveform may be a programmatic process that occurs in an automated fashion. In an alternate embodiment, the process may also involve human input in reviewing the selection of the target waveform and interpreting the results prior to completion of the analysis. In one embodiment, all of the analysis decisions are saved for future study in order to continually refine the stored waveform patterns 134. It should be noted that the analysis module 132 may be located on the local monitoring device or “off-site” at a remote location.

The results of the analysis performed by the analysis module 132 may be provided to one or more remotely located clinicians. In addition to displaying the captured breathing waveform, the analysis module 132 may also calculate and display the inspiratory/expiratory (I:E) ratio to the clinician. It should be appreciated that in some embodiments, the functionality attributed to the analysis module 132 may be split into additional modules without departing from the scope of the present invention. Depending upon the results of the analysis, the clinician may take a number of actions. The clinician may do nothing and continue to monitor the subject. Alternatively, the clinician may indicate to the patient that the incentive spirometry exercise is being performed incorrectly and needs to be adjusted in some manner. The embodiments of the present invention thus allow real-time monitoring and treatment of individuals performing incentive spirometry from a remote location.

In an alternative embodiment, the review of the generated waveform and response to the patient may be completely program driven. In such a case, the monitoring and response still occurs in real-time, but it occurs without human supervision.

Additionally, in one aspect of the present invention, a biofeedback module 106 may provide alternative sensory feedback designed to create an environment conducive to achieving or maintaining a desired respiratory status (for example to calm down a subject during an asthmatic event or prevent the onset of the event or to allow for easier monitoring). For example, biofeedback module 106 may provide visible displays, audible feedback such as music via audio module 140 or aromatic feedback via aroma dispensing module 144 designed to assist the monitored subject in achieving a desired breathing status. In one embodiment, the biofeedback may occur in the form of a voice giving algorithm-based guidance to a subject to attempt to lead the subject in a breathing exercise in order to bring the subject's breathing closer to a desired amplitude and thereby achieve a target waveform. By utilizing voice-based instruction, the subject may perform the breathing exercise with his or her eyes closed and avoid visual distractions that might otherwise be present.

In one embodiment, the analysis module 132 may report a significant discrepancy between the generated waveform and the target waveform that exceeds a pre-determined parameter. In such a circumstance, biofeedback module 106 may provide an intermediate waveform to monitored subject 120 rather than the target waveform in an attempt to incrementally adjust the breathing amplitude of the monitored subject. The intermediate waveform in such a situation may represent a more attainable goal to monitored subject 120 and its use may prevent the monitored subject from becoming alarmed (which is counter-productive) over the size of the difference between the generated and target waveforms. Biofeedback module 106 may provide a number of intermediate waveforms as appropriate for the monitored subject to attempt to replicate in order to incrementally move the monitored subject towards his or her target waveform. The embodiments of the present invention thus provide the ability to adjust real-time non-contact biofeedback based on the subject's actual response to the intervention.

The non-contact monitoring system may use radiated energy (e.g.: ultrasonic, radio frequency, infrared, laser, etc.) to identify respiratory waveforms in patients. The monitoring system may illuminate a subject in radiated energy and then detect the reflected radiated energy caused by respiratory functions. Of note, the breathing waveform can be captured through clothes and does not need a specific window to receive the necessary information to generate a breathing waveform. However, in one embodiment, a signal enhancer 122 may be utilized to augment the reflected signal. This may be in the form of a “relaxation patch” worn by the participant. The detected reflections are used to plot a two-dimensional waveform. The waveforms represent the rise and fall of a detected signal (the reflected energy) over time and are indicative of the small movements of the patient's chest, abdomen and/or other anatomical sites that are associated with respiratory function. Different implementations of the monitoring system use different forms of radiated energy (e.g.: laser, ultrasonic energy and radio frequency) to capture breathing waveforms for analysis. Following analysis, appropriate biofeedback is provided to the monitored subject.

One example of a suitable non-contact monitoring system that may be leveraged in conjunction with the embodiments of the present invention is described in U.S. Pat. No. 6,062,216 ('216 patent). As described in the '216 patent, a respiratory monitor may employ either ultrasonic or laser monitoring of an individual's breathing function by measuring changes in body position with respect to time. The device continuously and without the need for contact, monitors the individual's breathing function (and analyzes the measured waveform and identifies respiratory rate, apneic pauses, and obstructive breathing) and body movements. The '216 patent (the contents of which are hereby incorporated by reference) describes a monitoring system using laser energy or ultrasonic energy to monitor respiratory function so as to detect sleep apnea but may be adapted to perform the respiratory monitoring described herein. It should be appreciated that although the monitoring system of the '216 patent has been cited as an exemplary monitoring system which may be used in the present invention, other non-invasive monitoring systems utilizing laser or ultrasonic energy to detect respiratory waveforms may also be used and are within the scope of the present invention.

In one embodiment, the respiratory waveform detection module 102 may use ultrasound to perform the respiratory monitoring to establish the waveforms used in the present invention. Ultrasonic sound is a vibration at a frequency above the range of human hearing, in other words usually in a range above 20 kHz. In one embodiment, a shaped transducer in the monitoring system radiates a preferably continuous beam of ultrasound for example in the 25 kHz to 500 kHz range to illuminate a subject patient. A receiving transducer in the monitoring system of the present invention or transducer array develops one or more signals, which shift slightly from the incident frequency due to respiratory motion. The signal is then analyzed and plotted to generate a waveform, which may be compared against an appropriate benchmark. Appropriate adjustments are made by the monitoring system to account for the distance between the monitoring system and the subject as well as any environmental factors affecting the detection of the reflected energy.

In another embodiment, the monitoring system may use laser detection means as described in the '216 patent in place of ultrasonic energy. In such a case a laser illuminates the subject patient in a beam of light of a selected wavelength and the reflected energy, which varies, based on respiratory movements is traced so as to generate a waveform. Additionally, other embodiments utilizing infrared, radio frequency or other wavelength ranges in the electromagnetic spectrum may be employed in order to perform the non-contact monitoring and analysis of respiratory functions described herein.

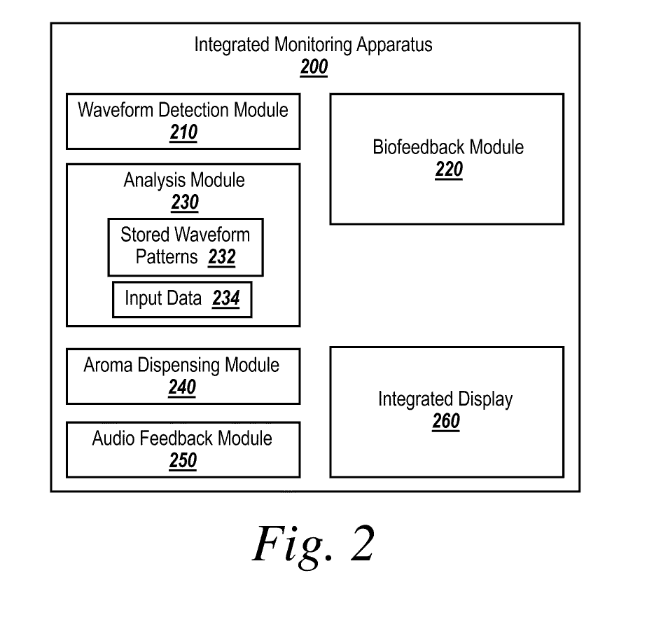

In one embodiment, the monitoring system described herein may be provided as an integrated monitoring apparatus rather than as separate components in multiple devices. FIG. 2 depicts an exemplary integrated monitoring apparatus 200 that includes most or all of the components of the monitoring system described in FIG. 1. The integrated monitoring apparatus 200 may include one or more waveform detection modules 210 such as respiratory waveform detection modules. The integrated monitoring apparatus 200 may also include biofeedback module 220 and analysis module 230. It will be appreciated that biofeedback module 220 and analysis module 230 may be combined into a single module or split into additional modules without departing from the scope of the present invention.

Analysis module 230 may include stored waveform patterns 232 as well as stored input data 234 specific to a monitored subject. In one embodiment, integrated monitoring apparatus 200 may also include an aroma dispensing module 240 and an audio module 250 for providing aromatic and audio feedback and an integrated display module 260 utilized to provide visual feedback to a monitored subject in the manner described herein. In other embodiments, integrated monitoring apparatus 200 may contain some but not all of the modules 240, 250 and 260 used to provide feedback and biofeedback. The aroma dispensing module 240 may include one or more stored scents that are designed to be soothing when inhaled and that are released into the monitored subject's environment at different times and in different amounts upon a signal being received from the biofeedback module 206. In an additional aspect of an embodiment of the present invention, the tactile, audible, visual and aromatic feedback may be dispensed as an adjunct to monitoring to prepare the subject for monitoring by creating a proper mood for monitoring prior to, or in addition to, any monitoring-based biofeedback being delivered.

In one exemplary embodiment, the integrated monitoring apparatus 200 may be provided via a portable device such as a mobile phone or smartphone, tablet computing device or laptop. For example, the mobile phone or smartphone, tablet computing device or laptop may be equipped with an ultrasound probe that is part of the device or connected via BLUETOOTH, or connected via a USB or other interface and that that is used to perform ultrasound monitoring. The detection and/or analysis modules described herein may be pre-installed or downloaded to the device. In one embodiment, portable devices such as a mobile phone or smartphone, tablet computing device or laptop display and speakers may be used to provide visual, audio and/or tactile feedback.

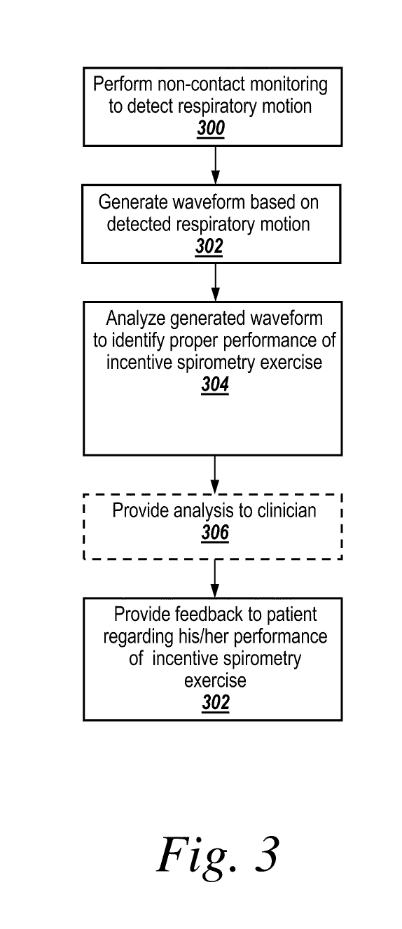

FIG. 3 depicts an exemplary sequence of steps performed by an embodiment of the present invention to monitor a patient's performance of incentive spirometry exercises. The sequence may begin by providing non-contact monitoring of a subject as described herein to detect respiratory motion (step 300). After data is gathered, a waveform is generated as a result of the monitoring process (step 302). The waveform is analyzed by the analysis module to identify whether the subject being monitored is properly inhaling during the exercise in a manner that will lead to increased lung volume (step 304). The analysis may include the generation of an I:E ratio from the generated waveform. The analysis may be provided to a clinician for further examination if a clinician is performing live supervision (step 306). The incentive spirometry system may then provide feedback to the user indicating to the patient whether or not the incentive spirometry exercise is being properly performed (step 308). In the event the patient is not performing the exercise correctly, visual or audible feedback may instruct the patient as to how to better perform the exercise.

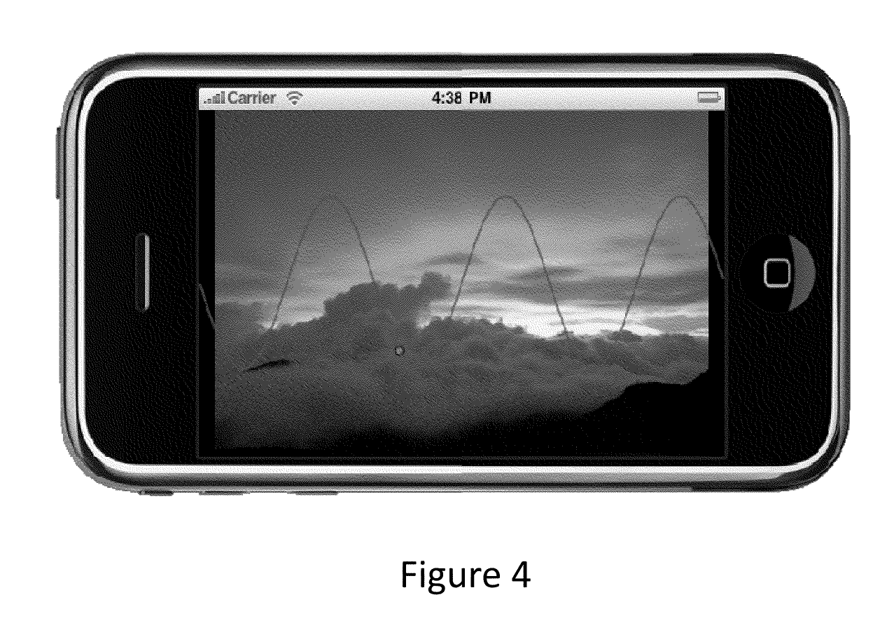

For example, FIG. 4 depicts an exemplary waveform that may be displayed to a patient to assist a user in performing incentive spirometry. The patient's waveform may be overlaid on the displayed target waveform so the patient knows whether or not to breathe deeper so as to increase the amplitude of his or her breathing. Alternatively, only the patient's waveform may be displayed and audible advice may be provided to the patient as to whether to breathe in a deeper or shallower manner.

As noted above, the embodiments of the present invention may be used to determine I:E ratios for a monitored subject. In one embodiment, the I:E ratio is calculated as the quotient of the duration of time that the target surface is moving away from the sensor (assumed to be expiration) and the duration of time the target is moving towards the sensor(assumed to be inspiration). Inspiration and expiration durations are monitored over several full breathing cycles so that the resulting measurement is an average. The ratio is displayed on the screen whenever a sufficient number of full breathing waveforms that pass a simple quality screen have been counted. The ratio is displayed as “1:X”—where X is the calculated quotient rounded to the nearest 0.25.

In one exemplary embodiment, the monitoring system monitors the physiologic parameters of a patient. Periodically a display screen alerts the subject that it is time for the relaxation or incentive spirometry therapy. The subject's breathing, and or heart rate and body movement waveforms are displayed. The subject can then alter his or her breathing to idealized patterns, which can be superimposed and displayed on the screen with the subject's actual waveforms. Of note, this time interval can be user set, set by a healthcare professional, or set based on monitored responses from the subject. For example, this session can be initiated by time intervals or based on the physiologic parameters being monitored. That is, for a subject whose respiratory amplitude is determined to be “too small” by the incentive spirometry algorithm, the window may come up sooner than the preset interval. The incentive spirometry system described herein thus also monitors for baseline respiratory and other parameters even when the incentive spirometry exercise is NOT being conducted. Conventional techniques have relied on intervening and changing physiologic parameters based on monitoring only when the subject is conscious and focused on the monitoring. In contrast, the background monitoring performed by the embodiments of the present invention is particularly beneficial to post-operative pain reduction since alterations in breathing, heart rate and the like have been shown to be important for relaxation and post-operative pain reduction. During the period when the incentive spirometry system is not being actively employed, should physiologic parameters be found to be out of range, soothing music, aromatic or visual therapy can also be automatically instituted.

The embodiments of the present invention may also be used for diagnostic purposes and tracking response to therapy as it allows for the continuous monitoring of subjects as they function in the environment of their computer or similar technology. This allows for halter-type assessment without the need for any physical contact with the subject being monitored.

The ability of the incentive spirometry system to store information allows an objective response to therapy, storage for medical records and is of possible importance for third-party reimbursement. Further, having objective and permanent records of responses to therapy may add to the attractiveness of the incentive spirometry technique and lead to better compliance with the regime. It should be understood that other physiologic parameters, the derivation of which are in the public domain (video, audio, etc) could be incorporated to add additional robustness to the proposed system. Further, though breathing, cardiac and body movement as described herein are derived through non-contact means, a contact system would also be obvious to one skilled in the art. It should also be noted that though a system is described that interfaces with any computing or laptop device, stand-alone technologies are also within the scope of the present invention.

In some embodiments, the monitoring and/or analysis modules may be deployed to the monitoring device as downloadable applets. For example, in one implementation, the monitoring and/or analysis modules may be downloaded to a smartphone or tablet computing device from a third party vendor, such as via the Apple iTunes® website.

The present invention may be provided as one or more computer-readable programs embodied on or in one or more physical mediums. The mediums may be a floppy disk, a hard disk, a compact disc, a digital versatile disc, a flash memory card, a PROM, an MRAM, a RAM, a ROM, or a magnetic tape. In general, the computer-readable programs may be implemented in any programming language. Some examples of languages that can be used include C, C++, C#, Python, FLASH, JavaScript, or Java. The software programs may be stored on, or in, one or more mediums as object code. Hardware acceleration may be used and all or a portion of the code may run on a FPGA, an Application Specific Integrated Processor (ASIP), or an Application Specific Integrated Circuit (ASIC). The code may run in a virtualized environment such as in a virtual machine. Multiple virtual machines running the code may be resident on a single processor.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described, the order of the acts may be modified in other implementations consistent with the principles of the invention. Further, non-dependent acts may be performed in parallel.

In addition, implementations consistent with principles of the invention can be implemented using devices and configurations other than those illustrated in the figures and described in the specification without departing from the spirit of the invention. Devices and/or components may be added and/or removed from the specifically disclosed implementations depending on specific deployments and/or applications.

We claim:

1. A non-contact monitoring system for monitoring incentive spirometry exercises, comprising:
a respiratory waveform detection module, the respiratory waveform detection module performing non-contact monitoring of a subject performing incentive spirometry exercises, the respiratory waveform detection module generating a waveform based on detected respiratory motion of the subject;
an analysis module programmatically analyzing the generated waveform based on a stored target waveform indicative of a target respiratory motion for an incentive spirometry exercise; and
a biofeedback module providing biofeedback to the subject to assist the subject in obtaining or maintaining the target waveform, the biofeedback based on a result of the analyzing of the generated waveform.

2. The system of claim 1, further comprising:
a display, the display used to provide the biofeedback in the form of a display of at least one of the generated waveform and the target waveform.

3. The system of claim 2 wherein the display of the generated waveform is accompanied by audible or visual instructions for the subject to increase or decrease a depth of breathing so as to attain the target waveform.

4. The system of claim 1, further comprising:
a display, the display used to provide the biofeedback in the form of a display of an intermediate waveform that represents a waveform between the generated waveform and the target waveform.

5. The system of claim 1, further comprising:
an auditory module that is used as an adjunct or to provide feedback in the form of audio transmissions detectable by the subject.

6. The system of claim 1 wherein the respiratory waveform detection module and the analysis module communicate over a network.

7. An integrated non-contact monitoring apparatus for monitoring incentive spirometry exercises, comprising:
a respiratory waveform detection module, the respiratory waveform detection module performing non-contact monitoring of a subject performing incentive spirometry exercises, the respiratory waveform detection module generating a waveform based on detected respiratory motion of the subject;
an analysis module programmatically analyzing the generated waveform based on a stored target waveform indicative of a target respiratory motion for an incentive spirometry exercise;
a biofeedback module providing biofeedback to the subject to assist the subject in obtaining or maintaining the target waveform, the biofeedback based on a result of the analyzing of the generated waveform; and
a display surface, the display surface used to provide the biofeedback in the form of at least one of a display of the generated waveform and the target waveform.

8. The apparatus of claim 7, further comprising:
an auditory module that is used to provide feedback in the form of audio transmissions detectable by the subject.

9. The apparatus of claim 7 wherein the respiratory waveform detection module monitors the subject using radiated energy.

10. The apparatus of claim 7 wherein the respiratory waveform detection module monitors the subject using ultrasound.

11. The apparatus of claim 7 wherein the respiratory waveform detection module monitors the subject using laser detection means.

12. The apparatus of claim 7 wherein the respiratory waveform detection module monitors the subject using infrared or radio frequency transmissions.

13. The apparatus of claim 7 wherein the display of the generated waveform is accompanied by audible or visual instructions for the subject to increase or decrease a depth of breathing so as to attain the target waveform.

14. A method for performing non-contact monitoring of incentive spirometry exercises, comprising:
performing non-contact monitoring of a subject performing an incentive spirometry exercise to detect respiratory motion of the subject;
generating programmatically a waveform based on the detected respiratory motion;
analyzing programmatically the generated waveform based on a stored target waveform indicative of a target respiratory motion for an incentive spirometry exercise; and
providing biofeedback to the subject to assist the subject in obtaining or maintaining the target waveform, the biofeedback based on a result of the analyzing of the generated waveform.

15. The method of claim 14, further comprising:
providing the biofeedback in the form of a display of at least one of the generated waveform and a target waveform.

16. The method of claim 14, further comprising:
conveying instructions for the subject to increase or decrease a depth of breathing so as to attain the target waveform.

17. The method of claim 14, further comprising:
providing the biofeedback in the form of a display of an intermediate waveform that represents a waveform between the generated waveform and the target waveform.

18. The method of claim 14, further comprising:
providing feedback in the form of an audio or visual transmission detectable by the subject.

19. A physical computer-readable medium holding computer-executable instructions for performing non-contact monitoring of incentive spirometry exercises, the instructions when executed causing one or more devices to:
perform non-contact monitoring of a subject performing an incentive spirometry exercise to detect respiratory motion of the subject;
generate programmatically a waveform based on the detected respiratory motion;
analyze programmatically the generated waveform based on a stored target waveform indicative of a target respiratory motion for an incentive spirometry exercise; and
provide biofeedback to the subject to assist the subject in obtaining or maintaining the target waveform, the biofeedback based on a result of the analyzing of the generated waveform.

20. The medium of claim 19 wherein the execution of the instructions further causes the one or more devices to:
provide the biofeedback in the form of a display of at least one of the generated waveform and the target waveform.

21. The medium of claim 20 wherein the execution of the instructions further causes the one or more devices to:
convey instructions for the subject to increase or decrease a depth of breathing so as to attain the target waveform.

22. The medium of claim 19 wherein the execution of the instructions further causes the one or more devices to:
provide the biofeedback in the form of a display of an intermediate waveform that represents a waveform between the generated waveform and the target waveform.

23. The medium of claim 19 wherein the execution of the instructions further causes the one or more devices to:
provide feedback in the form of an audio or visual transmission detectable by the subject.
